(19) United States
(12) Patent Application Publication
(10) Pub. No.: US 2009/0275928 A1
(43) Pub. Date: Nov. 5, 2009

(54) SUTURE-LESS LASER BLEPHAROPLASTY WITH SKIN TIGHTENING

(76) Inventors: Mark P. Solomon, Rydal, PA (US); Michael Moreno, Marklton, NJ (US); Michael M. Breen, River Forest, IL (US)

Correspondence Address: BIRCH STEWART KOLASCH & BIRCH, PO BOX 747, FALLS CHURCH, VA, 22040-0747, US

(21) Appl. No.: 12/434,222

(22) Filed: May 1, 2009

Related U.S. Application Data
(60) Provisional application No. 61/126,044, filed on May 1, 2008.

(51) Int. Cl. A61B 18/20

(52) U.S. Cl. 606/3; 606/9

(57) ABSTRACT

A laser is used to both remove fat and alter the collagen in the skin under the eye. Precisely controlling the heat rise in the dermis is critical to not only the fat removal and alteration of the collagen but also to prevent damage to surrounding tissue. The denatured collagen results in tightening of the skin, further enhancing the benefits of the procedure. The fat is removed by melting or vaporizing the fat deposits, depending on the laser strength. The method is minimally invasive with a short recovery time.

### CROSS REFERENCE TO RELATED APPLICATIONS

This application claims benefit of provisional application 61/126,044 filed May 1, 2008.

### BACKGROUND OF THE INVENTION

1. Field of the Invention

The present invention is directed to a method and apparatus for performing blepharoplasty.

2. Description of the Background Art

Lasers have been utilized in surgical techniques to treat skin conditions and remove fat deposits from patients. One example of the use of lasers is disclosed in U.S. Pat. No. 5,807,385 (Keller). The technique uses a quartz fiber to direct laser energy for the incision, division or resection of tissue. An endoscope may be utilized in conjunction with the quartz fiber to perform cosmetic surgical techniques. The laser energy may be used to perform a neck lift or reduce nasolabial folds.

U.S. Pat. No. 6,206,873 to Paolini et al. discloses a device and method for eliminating adipose layers with laser energy. An optical fiber is placed within a needle to deliver the laser energy. The laser energy is preferably pulsed with a wavelength between 0.75 and 2.5 micrometers, preferably between 0.8 and 1.1 micrometers.

Bulging of fatty deposits and loose skin appearance in the lower eye lids is caused by aging, environmental conditions, lifestyle and genetic disposition. Part of the natural aging process causing the lower eyelid to become puffy the condition is commonly referred to as “bags under the eyes.” Universally thought of as undesirable, many treatments have been developed to prevent, reverse or eliminate this condition.

Treatment options include radio frequency, surgery, carbon dioxide laser incision and numerous other treatments often provided in hospitals and at surgery centers. These treatments are often accompanied by a recovery period due to post treatment edema and bruising and most often do not include skin tightening. There are some techniques that are described as laser assisted eye lid treatment but these are believed to include laser in addition to other surgical techniques. The usual use of lasers in cosmetic procedures uses wavelengths which cannot be used near a patient's eye.

Tissue responds to heat depending on the temperature reached by the tissue. At 37-55 degrees Celsius, denaturation of the tissue occurs. Above 60 degrees Celsius, photo coagulation occurs but at 100-150 degrees Celsius, vaporization occurs and above 200 degrees Celsius, carbonization occurs. The range of 55-65 degrees Celsius is ideal for skin tightening based on the effect of collagen denaturation. Below 55 degrees Celsius has minimal results and over 70 degrees Celsius carries the potential for skin necrosis.

### SUMMARY AND OBJECTS OF THE INVENTION

There is a need in the art for a minimally invasive procedure for performing blepharoplasty in the area below the eye.

It is an object of an embodiment of the present invention to provide an apparatus and method of using lasers to treat the fat pads below the eye.

It is another object of an embodiment of the present invention to provide a method of treating the fat pads below the eye in a manner requiring a short recovery period.

It is a further object of an embodiment of the present invention to utilize laser energy to remove fat from the fat pads below the eye and provide skin tightening.

A laser is used to both remove fat and alter the collagen in the skin under the eye. Precisely controlling the heat rise in the dermis is critical to not only the fat removal and alteration of the collagen but also to prevent damage to surrounding tissue. The denatured collagen results in tightening of the skin, further enhancing the benefits of the procedure. The fat is removed by melting or vaporizing the fat deposits, depending on the laser strength. The method is minimally invasive with a short recovery time. The application of external laser energy in addition to the application under the skin further enhances the skin tightening effect

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a perspective view of the apparatus of the present invention;

FIG. 2 is a front view of an eye undergoing the procedure of the present invention; and

FIG. 3 is an enlarged view of the guide and fiber.

### DETAILED DESCRIPTION OF THE INVENTION

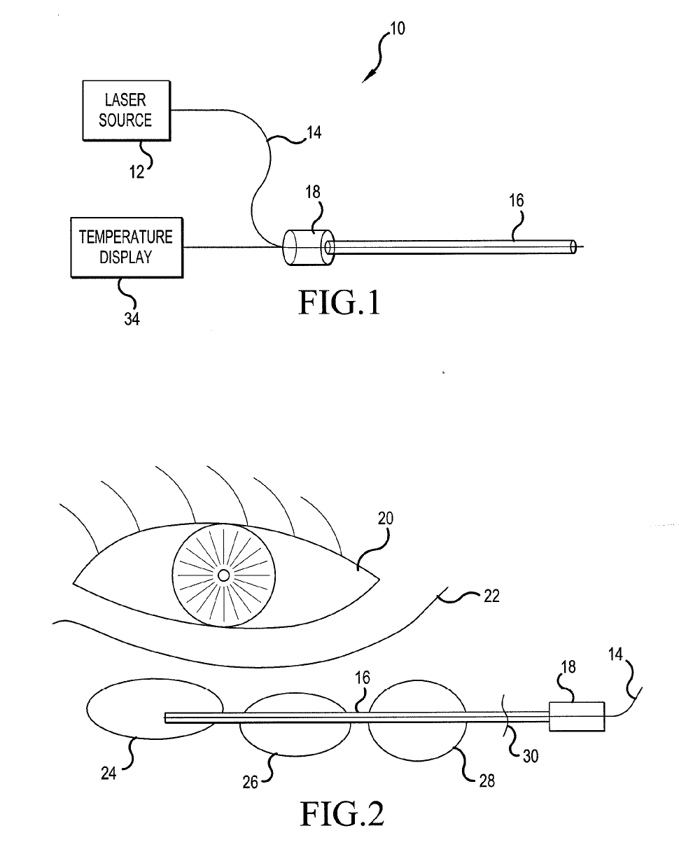

FIG. 1 illustrates an apparatus 10 used in the present invention. A laser source 12 provides laser energy having a wave length between 700 nm and 1470 nm, and may be applied continuously or pulsed. The range of 700-980 nm is preferred with the wavelength of 980 considered optimal. The laser wavelength may be delivered in either continuous or pulsed modes or both, with continuous being preferred. When pulsed, the pulse duration range may be between 0.2 to 6 seconds, preferably between 1-3 seconds, with a pulse interval between 100 milliseconds and 10 seconds. For either continuous or pulsed application, the energy level may be between milliwatts to 50 watts.

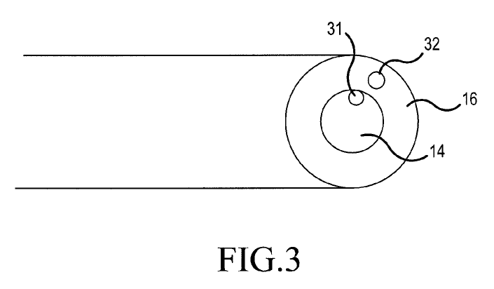

A laser fiber 14 delivers the laser energy to the area to be treated. The laser fiber has a coaxial structure and a diameter between 100 to 1000 microns. The laser fiber diameter controls the laser spot size created by the laser energy. In addition to delivering laser energy, the laser fiber may have a temperature sensor 31, seen in FIG. 3 to provide feedback to the operator through such means as a display 34, seen in FIG. 1. The laser fiber 14 is very flexible and cannot easily be moved to the desired position under an individual's skin due to this flexibility. For that reason, a guide 16 is used in conjunction with the laser fiber 14. The guide is rigid and hollow, preferably having an inner diameter matching the outer diameter of the laser fiber 14. Matching of the diameters in this manner ensures the correct positioning of the fiber as it is not allowed to move radially inside the guide but it possible to move the laser fiber axially within the guide once the guide is properly positioned. The guide can be made of any suitable material, such as metal, and can be a needle, endoprobe, catheter, trocar, cannula or any hollow fiber guide. The guide may be provided with a hand piece 18 enabling the operator to manipulate the guide and fiber. The guide may also have a temperature sensor 32, as seen in FIG. 3, to provide feedback to the operator through the display 34. Of course, both the fiber and guide may have a temperature sensor.

With the apparatus described, the method of using the apparatus to treat the area under the eye is explained with reference to FIG. 2, wherein a front view of a left eye 20 is depicted with the eye being retained within the orbital rim 22, the lower edge of which is shown in FIG. 2. In the example illustrated in FIG. 2, three fat deposits are disposed under the skin below the eye. The fatty deposits include the medial fat pad 24, center fat pad 26 and lateral fat pad 28. It is the fatty deposits that cause problems with puffiness around an individual's eye. The fat pads are located under the eye, between the orbital bone and muscles.

The method of performing blepharoplasty with skin tightening utilizing a laser starts with making a small incision 30 for inserting the guide 16 with the laser fiber 14 or other laser to be placed under the patient's skin and muscle. The incision 30 can be made in any appropriate location allowing the laser delivery device to access a fat pad under the eye. In one example, the incision can be made lateral to the lateral canthus, as depicted in FIG. 2. This allows access to all three fat pads through a single incision. Once the laser delivery device is placed under the patient's skin through the incision, the guide 16 is moved to the furthest fat pad, the medial fat pad 24. The guide is moved to the correct position and then the fiber inserted into the guide or the fiber may be placed in the guide before the guide in placed under the patient's skin. The surgeon may use any order of fat pads when treating the patient but using an incision at the outside edge of the lower eye lid and moving to the medial fat pad before moving to the central fat pad and then the lateral fat pad is the preferred order.

Once the guide 16 is embedded in the fat pad, the guide is moved in medial direction and dissection occurs along the orbital rim periosteum to the level of the lacrimal punctum. This is achieved by moving the proximal end of the guide 16 having the hand piece toward the patients head. A point on the guide spaced from the end of the guide forms a fulcrum with the orbital bone to cause the end of the guide to move away from the orbital bone, causing outward bulging of the patient's skin and muscles. At this point, the HeNe beam of the laser is visible through the skin, confirming the location of the end of the guide 16. Upon the application of laser energy, the fat is removed by being melted or vaporized. Depending on the level and duration of the laser energy, the amount of fat affected by the procedure can be controlled.

Removing a predictable percentage of fat and simultaneously heating the collagen fibers requires the use of thermal or electronic feedback to control the heat delivered to the target tissue while also monitoring the temperature of the surrounding non-target tissue. As mentioned previously, the laser fiber 14 or fiber guide 16 may include a temperature sensor or an external temperature sensor may be placed on the patient's skin to provide feedback to the operator. The appropriate amount of laser energy needs to be applied to the target tissue. The laser may have a wave length between 700 nm and 1470 nm, preferably 980 nm, and may be applied continuously or pulsed or both. When pulsed, the pulse duration range may be between 0.2 to 6 seconds, preferably between 1-3 seconds, with a pulse interval between 100 millisecomds and 10 seconds. For either continuous or pulsed application, the laser energy level may be between milliwatts to 50 watts.

The laser fiber 14 used to deliver the laser energy may have an inside diameter ranging from 400 to 1000 microns and may be delivered through a trocar, needle, endoprobe, cannula or catheter having an inner diameter substantially equal to the outer diameter of the laser fiber. Having the closely sized diameters increases the ease in positioning the laser fiber but is not necessary. The laser fiber delivers a laser spot having a spot size between 400 and 1000 microns.

When the desired amount of fat in the fat pad has been treated by the process, the guide 16 and laser fiber 14 are withdrawn to the next fat pad and the process is repeated. Once all fat pads are treated, the guide 16 and laser fiber 14 are withdrawn. The process is minimally invasive and requires a short recovery period. In addition to the removal of fat, the process also denatures collagen, having the added benefit of tightening skin in the area. The result is an enhanced appearance through a method that is inexpensive, requires a short recovery period and has superior results.

In addition to application of laser energy directly to the fat pad by the under skin positioning of the laser fiber, skin tightening can be further enhanced by applying laser energy externally to the lower eyelid. This can be done with the same type of laser fiber described above. The effect is enhanced skin tightening, faster healing, less bruising and swelling and a favorable cosmetic appearance.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1. A method of performing blepharoplasty, comprising:
inserting a laser fiber below the skin into at least one fat pad below an eye;
applying laser energy through the laser fiber to remove a percentage of the fat within the fat pad;
guiding the laser fiber with a guide.

2. The method of claim 1, wherein the inner diameter of the guide is equal to the outer diameter of the laser fiber.

3. The method of claim 1, further comprising:
inserting the laser fiber through three fat pads below the eye.

4. The method of claim 3, further comprising:
inserting the laser fiber laterally to the lateral canthus;
moving the fiber is a medial direction; and
pointing the laser fiber toward the skin surface.

5. The method of claim 4, wherein laser energy is first applied to a medial fat pad, secondly applied to a middle fat pad and lastly applied to a lateral fat pad.

6. The method of claim 1, further comprising:
monitoring the temperature of tissue about the fat pad being treated.

7. The method of claim 1, further comprising:
using a laser wavelength between 700 nm and 1470 nm.

8. The method of claim 1, further comprising:
using a continuous laser.

9. The method of claim 1, further comprising:
using a laser wavelength between 700 nm and 980 nm.

10. The method of claim 1, further comprising:
using a laser wavelength of 980 nm.

11. The method of claim 1, further comprising
applying laser energy externally to the lower eye lid

12. The method of claim 1, wherein the guide is at least one of a trocar, needle, endoprobe, cannula and catheter.

13. An apparatus for performing blepharoplasty below an individual's eye, comprising:
a laser source, the laser source supplying laser energy having a wavelength between 700 nm and 980 nm;
a laser fiber connected to the laser source; and
a guide having an outer diameter for enabling the guide to be inserted below an individual's eye, said laser fiber being positioned within the guide.

14. The apparatus of claim 12, wherein the guide is at lease one of a trocar, needle, endoprobe, cannula and catheter.

15. The apparatus of claim 12, wherein the laser fiber has an inside diameter between 400 to 1000 microns.

16. The apparatus of claim 12, wherein the laser fiber includes a temperature sensor.

17. The apparatus of claim 12, wherein the guide includes a temperature sensor.

18. The apparatus of claim 12, wherein the guide has an inner diameter substantially equal to the outer diameter of the laser fiber.
